(19) United States
(12) Patent Application Publication
(10) Pub. No.: US 2013/0316461 A1
(43) Pub. Date: Nov. 28, 2013

(54) SAMPLE ANALYZER, SAMPLE CONTAINER FOR QUALITY CONTROL, QUALITY CONTROL METHOD

(71) Applicant: SYSMEX CORPORATION, (US)

(72) Inventor: Kyozo FUJITA, Akashi-shi (JP)

(73) Assignee: SYSMEX CORPORATION, Kobe-shi (JP)

(21) Appl. No.: 13/839,515

(22) Filed: Mar. 15, 2013

(30) Foreign Application Priority Data
Mar. 30, 2012 (JP) 2012-080311

(51) Int. Cl. G01N 33/50

(52) U.S. Cl. CPC G01N 33/5005
USPC 436/63; 422/68.1; 422/547

(57) ABSTRACT

A sample analyzer comprises a sample analysis unit which analyzes a sample, a reading unit which reads information from a storage medium attached to a container that contains a quality control sample, a display, and a controller which displays on the display an analysis result obtained by another sample analyzer and read from the storage medium and an analysis result obtained by the sample analysis unit, when the storage medium stores the analysis result obtained by the other sample analyzer.

### FIELD OF THE INVENTION

The present invention relates to a sample analyzer which uses a quality control sample, container for quality control which contains a quality control sample, and a quality control method.

### BACKGROUND

Sample analyzers periodically perform measurements using the quality control sample to guarantee that accurate analysis results are output.

For example, Japanese Laid-Open Patent Application No. 2010-78477 discloses an automatic sample analyzer which analyzes a quality control sample, displays the allowed quality control range on a confirmation screen that shows the analysis results, and requires the user to determine whether the analysis results of the quality control sample is outside the allowed quality control range.

However, when a plurality of sample analyzers are installed in a single facility, for example, it is extremely important that the analysis results of the respective sample analyzers obtained using quality control samples are within the allowed range, it is also important that the analysis results among the sample analyzers are mutually close when the sample quality control sample is analyzed by the respective sample analyzers. Conventionally, therefore, uniformity of analysis results among sample analyzers has been verified by printing analysis results of a quality control sample and comparing the respective analysis results from the several sample analyzers. This work is extremely difficult, however.

### FIELD OF THE INVENTION

The scope of the present invention is defined solely by the appended claims, and is not affected to any degree by the statements within this summary.

A first aspect of the present invention is a sample analyzer, comprising a sample analysis unit which analyzes a sample, a reading unit which reads information from a storage medium attached to a container that contains a quality control sample, a display, and a controller which displays on the display an analysis result obtained by another sample analyzer and read from the storage medium and an analysis result obtained by the sample analysis unit, when the storage medium stores the analysis result obtained by the other sample analyzer.

A second aspect of the present invention is a sample container for quality control, comprising a container for holding a quality control sample, and a storage medium which is readable and writable for analysis information of the quality control sample held in the container.

A third aspect of the present invention is a quality control method for performing quality control of a sample analyzer, comprising reading from a storage medium a first analysis result obtained by a first sample analyzer analyzing a quality control sample held in a container, obtaining a second analysis result from a second sample analyzer which analyzes the quality control sample held in the container and is different from the first sample analyzer, and displaying the first analysis result and the second analysis result on a display of the second sample analyzer.

### BRIEF DESCRIPTION OF THE DRAWINGS

FIG. 1 is a perspective view showing an external view of an embodiment of the sample analyzer;

FIGS. 2A, 2B, and 2C show the structures of the rack (FIG. 2C), sample container (FIG. 2A), and quality control sample container (FIG. 2B) of the embodiment;

FIG. 3 is a schematic view showing the structures of the transporting unit and measurement unit of the embodiment viewed from above;

FIG. 4 briefly shows the structures of the transporting unit and the measurement unit of the embodiment;

FIG. 5 briefly shows the structure of the fluid circuit of the measurement unit of the embodiment;

FIG. 6 briefly shows the structure of the RFID reader/writer and RFID tag of the embodiment;

FIG. 7 briefly shows the structure of the information processing unit of the embodiment;

FIG. 8 shows an example of the usage of the sample analyzer of the embodiment;

FIGS. 9A, 9B, 9C, 9D, 9E and 9F show the data structure stored on the RFID tag, and the data structure stored on the hard disk of the sample analyzer of the embodiment;

FIG. 10 is a flow chart showing the process of comparing the quality control measurements of the embodiments;

FIG. 11 is a flow chart showing the process of comparing the quality control measurements of the embodiments;

FIGS. 12A, 12B, and 12C show a flow chart of the warning message process, the leader chart display process, and the time series chart display process, respectively, of the embodiment;

FIG. 13 shows the warning message display screen of the embodiment;

FIG. 14 shows the leader chart display screen of the embodiment;

FIG. 15 shows the time series chart display screen of the embodiment; and

FIG. 16 shows an example of the usage of the sample analyzer of the embodiment.

### DETAILED DESCRIPTION OF THE PREFERRED EMBODIMENTS

The preferred embodiments of the present invention will be described hereinafter with reference to the drawings.

The present embodiment applies the invention to a sample analyzer for performing examinations and analysis of blood.

The sample analyzer of the embodiment is described below referring to the drawings.

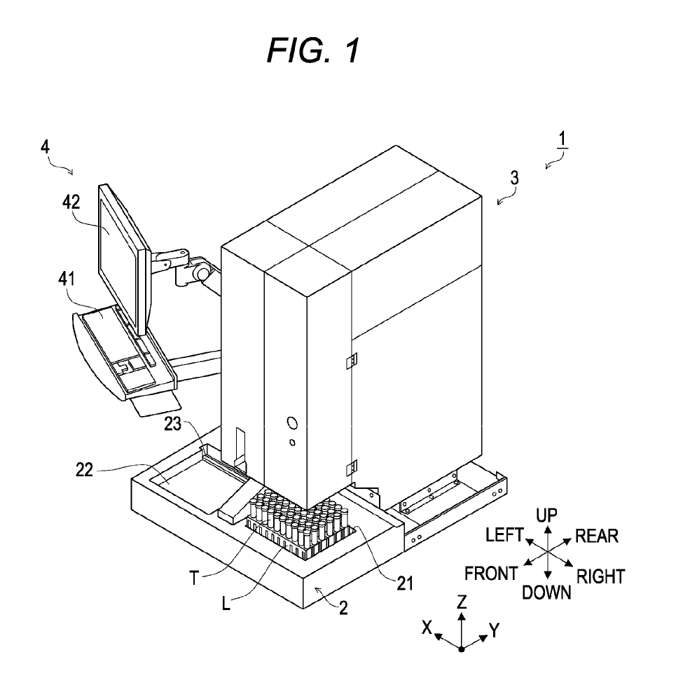

FIG. 1 is an exterior perspective view of a sample analyzer 1. The sample analyzer 1 of the present embodiment is configured by a transporting unit 2, measurement unit 3, and information processing unit 4.

The transporting unit 2 is disposed in front of the measurement unit 3, and is configured by a right table 21, left table 22, and a rack transporter 23 connecting the right table 21 and the left table 22. The right table 21 and the left table 22 can accommodate a plurality of racks L that are capable of holding ten sample containers T.

The transporting unit 2 can hold a rack L installed on the right table 21 by the operator. The transporting unit 2 transports the rack L held on the right table 21 to a predetermined position of the rack transporter 23 to supply the sample containers T to the measurement unit 3. The transporting unit 2 then transports the rack L on the rack transporter 23 to the left table 22.

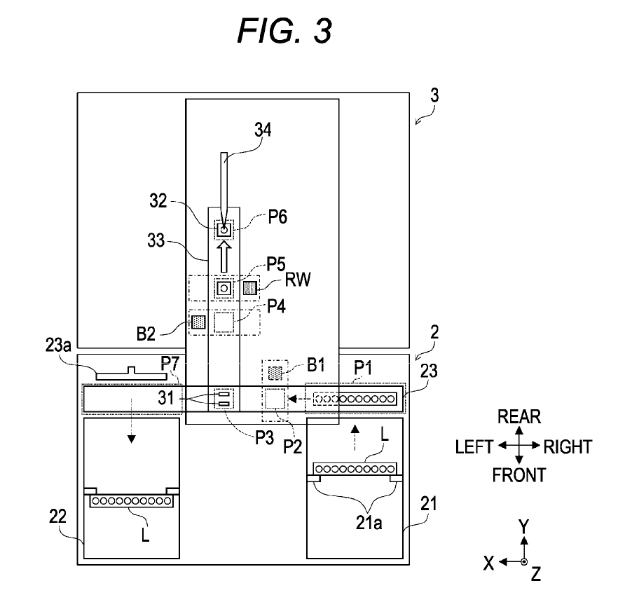

In the present embodiment, the containers in the rack L are picked up for processing by the measurement unit 3 at the take-up position P3 (refer to FIG. 3) on the rack transporter 23.

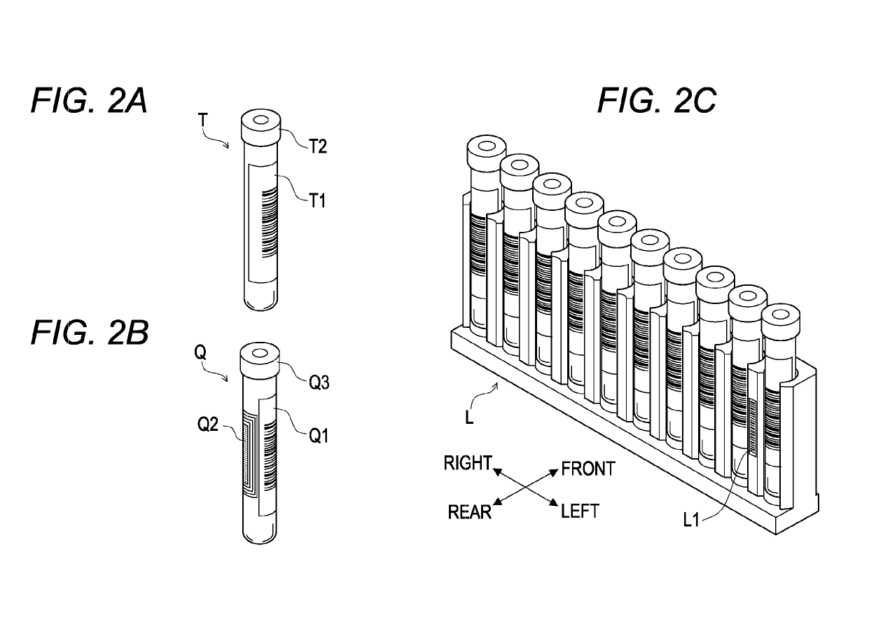

FIGS. 2A, 2B, and 2C, respectively show the structures of the sample container T, quality control sample container Q, and the rack L. FIG. 2A and FIG. 2B are perspective views showing the exteriors of the sample container T and the quality control sample container Q; FIG. 2C is a perspective view showing the exterior of a rack L holding ten sample containers T. Note that FIG. 2C shows the directions (front/back, left/right of FIG. 1) when the rack L is placed on the transporting unit 2.

Referring to FIG. 2A, the sample container T is a tube-like container, open at the top end, formed of transparent synthetic resin or glass. A barcode label T1 is adhered to the side surface of the sample container T. A barcode including the sample ID is printed on the barcode label T1. The sample container T contains a blood sample of whole blood collected from a patient, and the opening at the top end is sealed with a rubber cap T2.

Referring to FIG. 2B, the quality control sample container Q is a tube-like container, open at the top end, and formed of synthetic resin or glass. A barcode label Q1 and an RFID tag Q2 are adhered to the side surface of the quality control sample container Q.

A barcode containing a quality control ID representing the quality control sample, and different than a sample ID, is printed on the barcode label Q1. Quality control is a management method for determining whether a certain degree of analytical precision is obtained by measuring a quality control sample containing a known amount of a predetermined substance, and monitoring the measurement results. Control blood containing a known amount of predetermined blood components, for example, can be used as the quality control sample. Red blood cells including reticulocytes and nucleated red blood cells, and white blood cells including lymphocytes, monocytes, neutrophils, eosinophils, basophils may be used as the predetermined blood components The components may be obtained by preparing blood collected from a living body, or may be artificially prepared imitation components. If the prepared quality control sample is measured to obtain the predetermined measurement result and the measurement result exceeds the set range of a target value, it is determined that an abnormality has occurred in wither the sample analyzer 1 or in the quality control sample. Quality control is usually performed 1 to 3 times each day. Note that since quality control samples are expensive the use of a single quality control sample should be considered for a plurality of sample analyzers 1.

The RFID tag Q2 is a readable/writable non-contact type electronic tag that is prewritten with information of the quality control sample itself such as the sample name, lot number, target value, expiration date and the like. In addition to the information of the quality control sample itself, the RFID tag Q2 has an empty area for storing the results obtained by measuring the quality control sample. Note that the barcode label Q1 and the RFID tag Q2 are respectively placed separately, the barcode may also be printed, for example, on the surface of the RFID tag. The quality control sample container Q contains a blood sample prepared for use in quality control, and the open top of the container is sealed by a rubber cap Q. The quality control sample container Q has the same shape and size as the sample container T.

Referring to FIG. 2C, a barcode label L1 is adhered to the back side of the rack L. A barcode including the rack ID is printed on the barcode label L1. The rack L has holders capable of vertically holding ten sample containers T and quality control sample containers Q.

Returning now to FIG. 1, during measurement of a sample, the measurement unit 3 performs processing of the sample container T on the rack transporter 23 in front of the measurement unit 3. That is, the measurement unit 3 removes the sample container T from the rack L via the hand part 31 (refer to FIG. 3) at the take-up position P3 (refer to FIG. 3) of the rack transporter 23, and moves the sample container T into the measurement unit 3, and the sample within the sample container T then is measured in the measurement unit 3. When the measurement is completed, the measurement unit 3 returns the sample container T back to the original holder of the sample rack L. The measurement of the quality control sample when the quality control sample is measured is performed identically to the measurement of a blood sample.

The information processing unit 4 has an input part 41 and a display part 42. The information processing unit 4 is connected via a communication network so as to be capable of communicating with the transporting unit 2 and measurement unit 3. The information processing unit 4 controls the operation of the transporting unit 2 and the measurement unit 3, and analyzes the measurement data received from the measurement unit 3. The information processing unit 4 also shows predetermined information, such as messages, on the display part 42.

FIG. 3 is a schematic view showing the structures of the transporting unit 2 and measurement unit 3 viewed from above.

The barcode information reading operation is described first referring to FIG. 3.

The rack L loaded in the right table 21 is moved to the feed position P1 on the right end of the rack transporter 23 by the rack mover 21a pushing the front side of the rack L. The rack L placed at the feed position P1 is then moved leftward by a belt (not shown in the drawing) of the rack transporter 23.

A barcode reader B1 is installed near the center of the rack transporter 23. When the holder of the rack L is disposed at the barcode reading position P2 in front of the barcode reader B 1, whether a container (sample container T or quality control sample container Q) is held in the holder is determined based on the detection result of a sensor that detects the presence of the container.

When a container is held in the holder, the barcode reader B1 reads the sample ID from the barcode label T1 of the sample container T and the barcode label Q1 of the quality control sample container Q. When the barcode label L1 of the rack L is disposed in front of the barcode reader B1, the barcode reader B1 reads the rack ID from the barcode label L1 of the rack L.

Hence, the barcode information of the rack L, the information of the presence of a container in all holders of the rack L, and the barcode information of the containers held in the rack L are obtained.

The operation for supplying the sample container T and the quality control sample container Q of the rack L to the measurement unit 3 is described below.

When reading the barcode information as mentioned above, the container (sample container T or quality control sample container Q) in the holder of the rack L is disposed at the take-up position P3 of the measurement unit 3. The hand part 31 is provided in the measurement unit 3 so as to be movable in vertical directions (Z-axis direction) at the take-up position P3.

When the container is disposed at the take-up position P3, the sample container T is gripped by the hand part 31 and moved in the upward direction (positive Z-axis direction). The hand part 31 moves the container in a pendulum-like fashion to mix the sample. At this time the container placement part 32 is moved above the take-up position P3. When mixing is completed, the hand part 31 moves in a downward direction (negative Z-axis direction) and the container held by the hand part 31 is set in the container placement part 32.

The sample placement part 32 is thereafter moved to the barcode reading position P4 by the container transporter 33, and the barcode reader B2 reads the barcode to verify the container.

When the container set in the container placement part 32 is a quality control sample container Q, the container placement part 32 is moved to the RFID tag reading position P5 by the container transporter 33. When the container placement part 32 is disposed at the RFID tag reading position P5, the RFID reader/writer RW reads the RFID tag Q2 of the quality control sample container Q. The RFID reader/writer RW has the function of reading and writing the content of the RFID tag Q2 adhered to the quality control sample container Q via non-contact radio waves. The container placement part 32 is then moved to the aspirating position P6 directly below the piercer 34 by the container transporter 33.

When the container set in the container placement part 32 is a sample container T, the container placement part 32 is not disposed at the RFID tag reading position P5, but is moved to the aspirating position P6.

When the container placement part 32 is disposed at the aspirating position P6 directly below piercer 34, the piercer 34 moves downward to aspirate the sample from the sample container T disposed at the aspirating position P6.

When a sample container T is in the container placement part 32 and aspiration of the sample is completed by the piercer 34, the container placement part 32 is moved forward and repositioned at the take-up position P3.

When a quality control sample container Q is set in the container placement part 32 and aspiration of the sample is completed by the piercer 34, the container placement part 32 remains on standby at that position until the analysis results of the sample have been obtained. When the analysis results of the sample have been obtained, the container placement part 32 is moved forward and repositioned at the RFID tag reading position P5. When the container placement part 32 is disposed at the RFID tag reading position P5, the RFID reader writer RW writes the analysis results of the quality control sample to the RFID tag Q2 of the quality control sample container Q. When the quality control sample analysis results have been written, the container placement part 32 is moved to the take-up position P3.

The container at the take-up position P3 is lifted from the container placement part 32 by the hand part 31. In this state, the container placement part 32 is moved backward. The hand part 31 is then moved downward (negative Z-axis direction), and the container is returned to the original holder of the rack L disposed on the rack transporter 23. Thereafter, the container in the next holder of the rack L is supplied to the measurement unit 3. When all containers of the rack L have been measured, the rack L is moved to the recovery position P7. The rack L is then discharged to the left table 22 by the rack pusher 23a.

Note that although an RFID reader/writer RW having the function of reading and writing the RFID tag is used in the present embodiment, an RFID reading having only a reading function and an RFID writer having only a writing function also may be used. In this case the RFID reader is arranged between the feed position P1 and the aspirating position P6, and the RFID writer is arranged between the aspirating position P6 and the recovery position P7. Typically, since measuring a sample requires a certain amount of time, the RFID writer is preferably arranged between the take-up position P3 and the recovery position P7 of the rack transporter 23. In this case, during the measurement operation but after the quality control sample is aspirated by the measurement unit 3, the quality control sample container Q is returned to the rack L so the next container can be taken into the measurement unit 3 to smoothly perform the measurement operation of the subsequent sample.

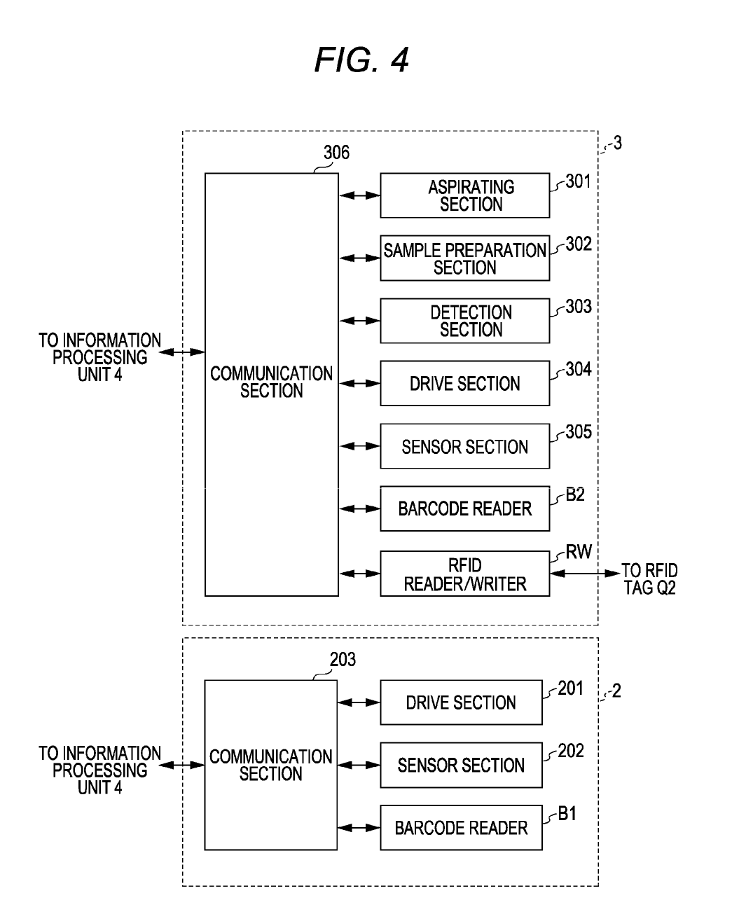

FIG. 4 briefly shows the structures of the transporting unit 2 and the measurement unit 3.

The transporting unit 2 has a drive section 201, sensor section 202, barcode reader B1, and communication section 203.

The drive section 201 includes a device for moving the rack L within the transporting unit 2, and the sensor section 202 includes sensors for detecting the rack L at predetermined positions on the transport path of the transporting unit 2. The barcode reader B1 reads the barcode labels adhered to the rack L, the sample container T, and the quality control sample container Q, respectively.

The communication section 203 is connected to the information processing unit 4 and is capable of communication therewith. Each section in the transporting unit 2 is controlled by the information processing unit 4 through the communication section 203. Signals output from the various sections in the transporting unit 2 are also transmitted to the information processing unit 4 through the communication section 203.

The measurement unit 3 has an aspirating section 301, sample preparing section 302, detecting section 303, drive section 304, sensor section 305, barcode reader B2, RFID reader/writer RW, and communication section 306.

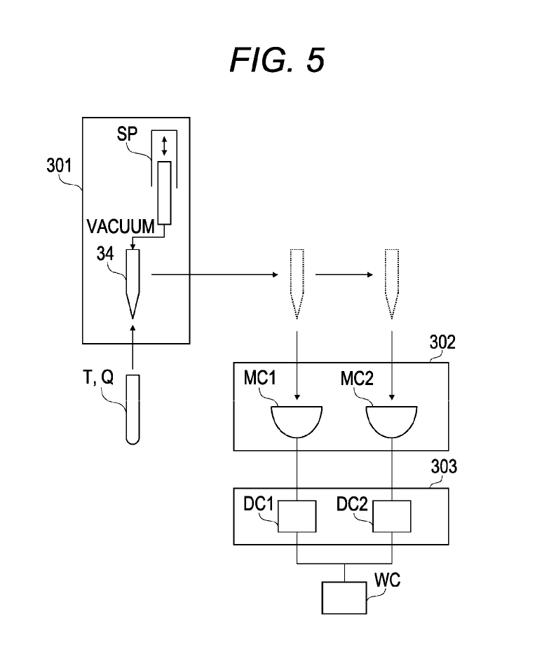

FIG. 5 briefly shows the fluid circuit of the measurement unit 3.

The aspirating section 301 includes the piercer 34 for aspirating the quality control sample contained in the quality control sample container Q and the sample contained in the sample container T that is transported within the measurement unit 3, and a syringe pump SP for producing a negative pressure to the piercer. The sample preparing section 302 has a reaction chamber MC1 for preparing samples for measuring red blood cells and platelets, and a reaction chamber MC2 for preparing samples for measuring white blood cells. The detecting section 303 has an electrical resistance type detector DC1 for measuring red blood cells and platelets, and optical type detector DC2 for optically measuring white blood cells, nucleated red blood cells, reticulocytes and the like. The measurement unit 3 also has a waste fluid chamber WC for storing waste fluids.

When measuring a sample and quality control sample, the aspiration section 301 aspirates the sample through the piercer 34 by inducing a negative pressure in the piercer 34 via the syringe pump SP, and discharges the sample to the reaction chambers MC1, MC2. The sample preparing section 302 stirs and mixes the sample and reagent within the reaction chamber MC1 to prepare a sample to be used for measuring red blood cells and platelets. The sample preparing section 302 also stirs and mixes the sample and reagent within the reaction chamber MC2 to prepare a sample for measuring white blood cells and a sample for measuring reticulocytes. The sample prepared in the reaction chamber MC1 is moved to the electrical resistance type detector DC1 through a flow path, and the sample prepared in the reaction chamber MC2 is moved to the optical type detector DC2 through a flow path. The detecting section 303 detects the optical information (side fluorescent light signals, forward scattered light signals, and side scattered light signals) from the white blood cells, nucleated red blood cells, and reticulocytes in the sample as sample data via the optical type detector DC2 as sample data. The detecting section 303 also detects the electrical information from the red blood cells and platelets in the sample as sample data via the electrical resistance type detector DC1. The samples that have passed through the detecting section 303 are then moved to the waste liquid chamber WC through a flow path.

Returning to FIG. 4, the drive section 304 includes a mechanism to transport the sample container T and the quality control sample container Q within the measurement unit 3. The sensor section 305 includes sensors to detect the sample container T and the quality control sample container Q at predetermined positions on the transport path within the measurement unit 3. The barcode reader B2 reads the barcode label adhered to the sample container T and the quality control sample container Q transported in the measurement unit 3.

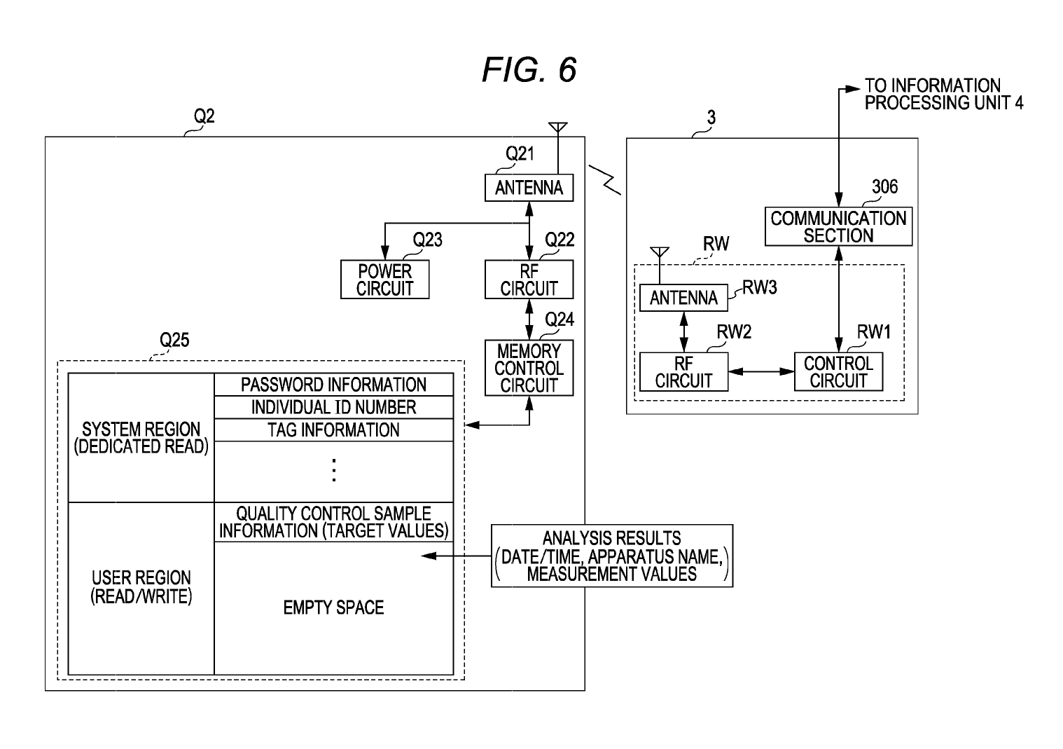

FIG. 6 briefly shows the RFID reader/writer RW and the RFID tag Q2. Note that FIG. 6 schematically shows part of the measurement unit 3 and the RFID tag Q2, and conceptually shows the empty addresses of memory Q25 of the RFID tag Q2.

The RFID reader/writer RW has a control circuit RW1, RF circuit RW2, and antenna RW3. The control circuit RW1 includes a CPU and memory, and generates read and write commands for the RFID tag Q2 in accordance with the control signals receives from the information processing unit 4. The RF circuit RW2 modulates between radio waves and transmission data. The antenna RW3 generates radio waves, and supplies power to the RFID tag Q2 and for communication between the RFID reader/writer RW and the RFID tag Q2.

The RFID tag Q2 is a passive type RFID tag without it's own power source, and has an antenna Q21, RF circuit Q22, feed circuit Q23, memory control circuit Q24, and memory Q25. The antenna Q21 receives radio waves, and outputs the received radio waves to the RF circuit Q22 and feed circuit Q23. The RF circuit Q22 performs modulates between radio waves and transmission data. The feed circuit Q23 converts the radio waves from the antenna Q21 to a direct current power voltage and supplies the current within the RFID tag Q2. The memory control circuit Q24 performs data reading and writing processes to the memory Q25 in accordance with the read and write commands received from the RFID reader/writer RW.

The memory Q25 has address space of a predetermined memory capacity, and has a read only system region and a readable/writable user region. Security information such as a password, a number (unique ID) identifying the individual RFID tag, and tag-specific information usable by the tag manufacturer are prewritten to the system region when the RFID tag is fabricated.

The lot number, target values, and information on the quality control sample contained in the container are written to the user region. The memory Q25 of the RFID tag Q2 is of sufficient size to allow writing information of the quality control sample consisting of a plurality of items.

The user region also contains empty space for storing analysis results (date/time, device name, measurement values) of the quality control sample measured by the measurement unit 3. The empty space is of sufficient size to hold a predetermined number of analysis results for quality control samples. When the empty region becomes insufficient due to storing analysis results, the information processing unit 4 controls the RFID reader/writer so as to sequentially overwrite or delete from the oldest analysis result to ensure there is empty space in the memory Q25 of the RFID tag Q2. Note that in this case the information processing unit 4 and the RFID reader/writer RW store analysis results and perform deletions so that the quality control sample information (lot number, target value) is not overwritten or deleted. In this way the area in the user region which stores the quality control sample information is only read, whereas the quality control analysis results are written and deleted in the other empty area as appropriate.

The communication section 306 is connected to the information processing unit 4 and is capable of communication therewith. Each section of the measurement unit 3 is controlled by the information processing unit 4 through the communication section 306. Signals output from the various sections in the measurement unit 3 are also transmitted to the information processing unit 4 through the communication section 306.

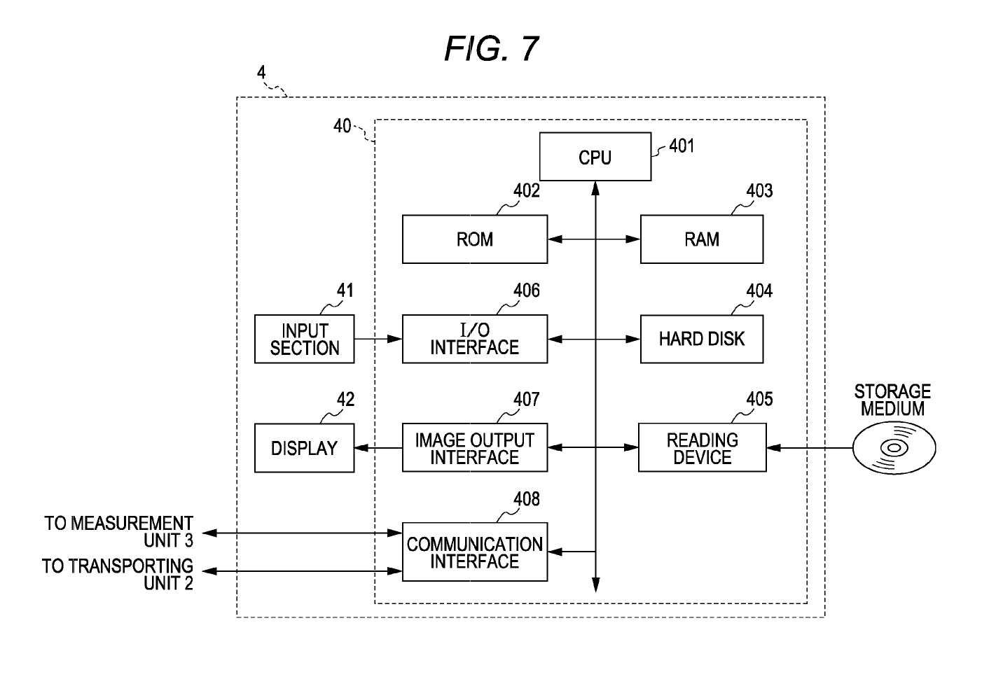

FIG. 7 shows the essential structure of the information processing unit 4.

The information processing unit 4 is configured by a personal computer having a main body 40, input section 41, and display section 42. The main body 40 has a CPU 401, ROM 402, RAM 403, hard disk 404, reading device 405, I/O interface 406, image output interface 407, and communication interface 408.

The CPU 401 is capable of executing a computer program stored in the ROM 402 and a computer program loaded in the RAM 403. The RAM 403 is used when reading the computer program stored in the ROM 402 and recorded on the hard disk 404. The RAM 403 is also used as the work area of the CPU 401 when the CPU 401 executes the computer programs.

An operating system and application programs, as well as the data used when executing the operating system and application programs that are executed by the CPU 401, are installed on the hard disk 404. That is, the hard disk 404 stores programs for analyzing the sample data transmitted from the measurement unit 3 and generating measurement results such as the red blood cell count and white blood cell count, and showing results on the display section 42 based on the generated measurement results.

Measurement order, recording date/time information, and status information are stored on the hard disk 404. Measurement orders are information including various items as well as the sample ID, and measurement items associated with the sample ID. The recording date and time is information representing the date and time the measurement order was recorded, and is stored in memory associated with each measurement order. The status information is information indicating whether the measurement was completed based on the measurement order, and is stored in memory associated with each measurement order.

The reading device 405 is a CD drive or DVD drive capable of reading computer programs and data recorded on a recording medium. The I/O interface 406 is connected to the input section 41 configured by a mouse and keyboard, and the user uses the input section 41 to input instructions and data to the information processing unit 4. The image output interface 407 is connected to the display section 42 configured by a display of some type, and the image output interface 407 outputs image signals corresponding to the image data to the display 42.

The display section 42 displays images based on the input image signals. Various types of program screens are shown on the display section 42. Data transmission and reception is possible with the transporting unit 2, and measurement unit 3 through the communication interface 408.

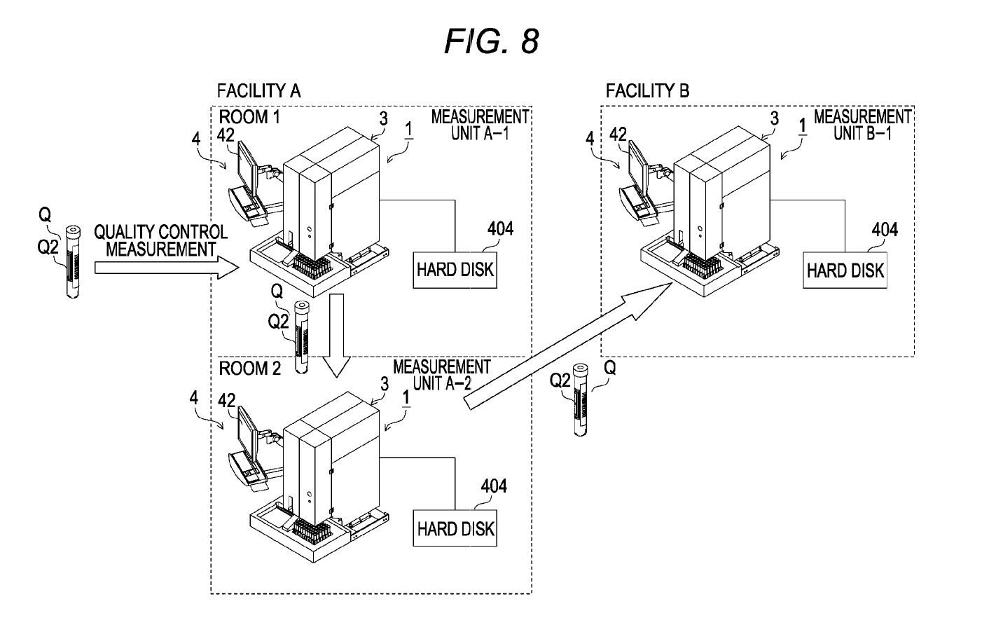

FIG. 8 shows an example of the usage conditions of the sample analyzer 1.

The sample analyzer 1 can be assumed to be used in a plurality of rooms in a plurality of institutions as shown in the diagram. Since the quality control sample is expensive as mentioned above, a single quality control sample may be used jointly by a plurality of apparatuses. In the example of FIG. 8, a measurement unit A-1 is arranged in room 1 of facility A, and a measurement unit A-2 is arranged in room 2, which is separate from room 1, of facility A. A further measurement unit B-1 is arranged in facility B, which is near facility A. For example, one quality control sample container Q may be used jointly by the measurement units A-1, A-2, B-1. Note that the measurement units A-1, A-2, and B-1 are respectively situated at separated locations, and are not connected so as to be capable of intercommunication.

The method of comparing quality control analysis results obtained by the several sample analyzers is described below.

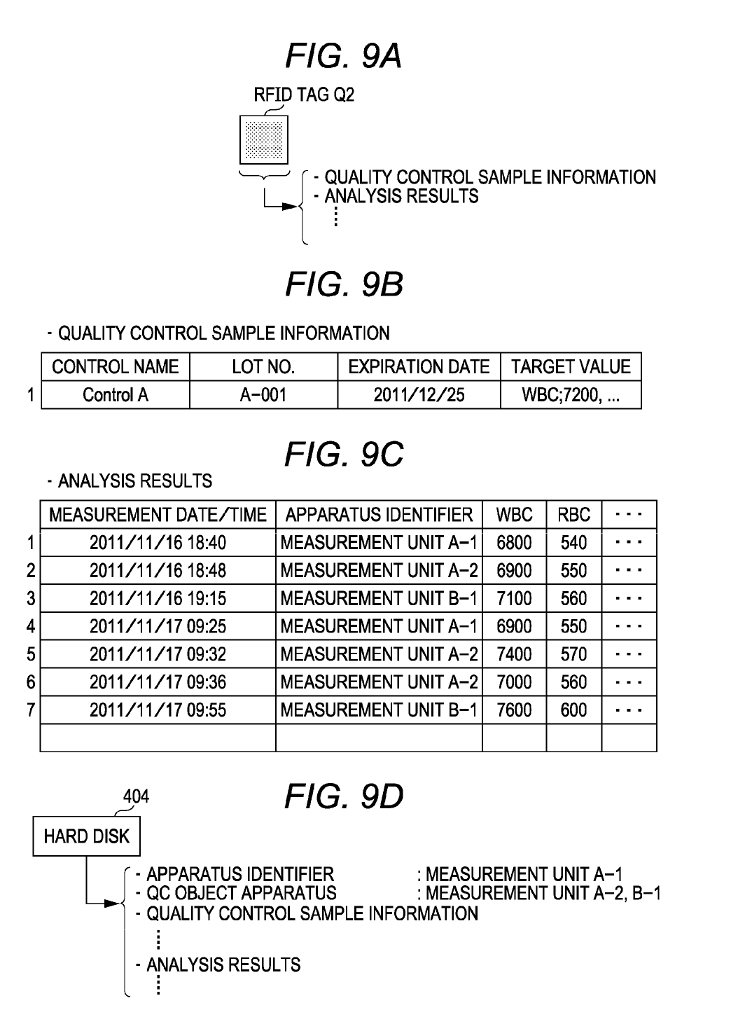

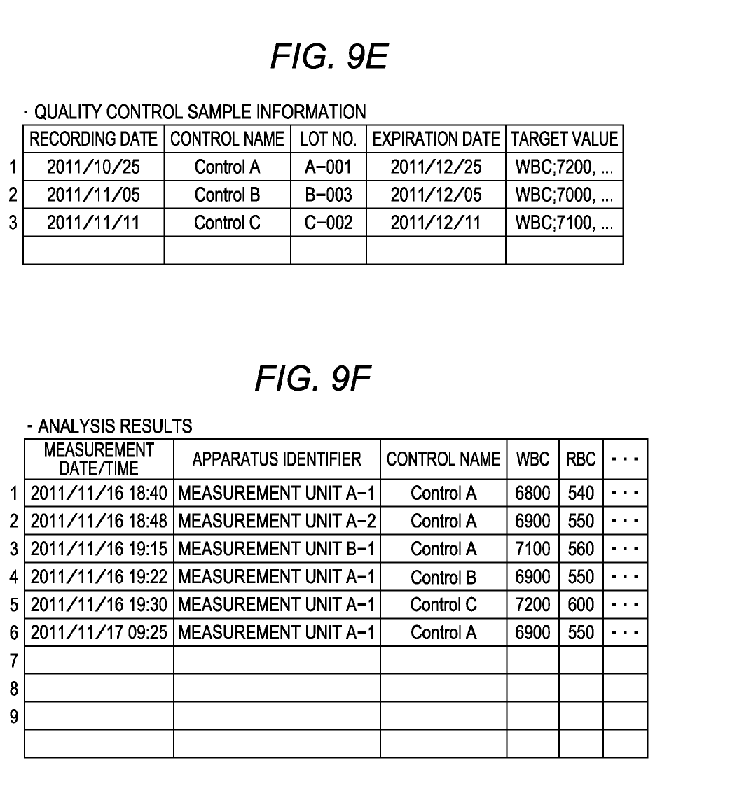

FIGS. 9A, 9B, and 9C are conceptual diagrams showing the data structure of data stored in the RFID tag Q2 of the quality control sample container Q. FIGS. 9D, 9E, and 9F are conceptual diagrams showing the data structure of data stored on the hard disk 404 of the sample analyzer 1. Note that FIGS. 9B, 9C, 9E, and 9F show predetermined values stored when the quality control sample measurement was performed in the sequence of measurement unit A-1, A-2, B-1. These values are ordered based on the measurement date/time, in ascending sequence based on the recording day, and do not necessarily indicate the sequence in which they were written.

Referring to FIG. 9A, the quality control sample information and a plurality of analysis results are included in the RFID tag Q2 of the quality control sample container Q. The quality control sample information includes control name items, lot number items, expiration date items, and target value items. A name which identifies the quality control sample is stored in the control name item. The manufacturer number of the quality control sample, which was assigned during fabrication, is stored in the lot number item. The year/month/day of the expiration date of the quality control sample, which represents the period of effective period of quality control, is stored in the expiration date item. Target values for measurement items (WBC, RBC and the like) when measuring the quality control sample are stored in the target value item. Note that the target value is determined at the time of manufacture of the quality control sample and is different from the lot number. These items and set values are pre-stored in the RFID tag Q2 during the manufacture of the quality control sample.

As shown in FIG. 9C, the analysis results include the measurement date/time item, apparatus identifier name item, and each measurement item (WBC, RBC). The date and time at which the quality control sample is measured is stored in the measurement date/time item. The name of the measurement unit performing quality control is stored in the apparatus identifier name item. The analysis results of the various measurement items are stored in the measurement items (WBC, RBC and the like). The quality control sample is used jointly by a plurality of sample analyzers as described above, and the analysis results of a plurality of analyses are stored for the three measurement units A-1, A-2, B-1.

Referring to FIG. 9D, the apparatus identifier, quality control object device, plurality of quality control sample information, and plurality of analysis results are stored on the hard disk 404 of the sample analyzer 1. The name of the measurement unit performing quality control is stored in the apparatus identifier. The names of the measurement units for which the quality control analysis are being compared for the same quality control sample are stored in the quality control object apparatus. For example, when measurement unit A-1 is used in room 1 of facility A shown in FIG. 8, the name measurement unit A-1 is stored in the apparatus identifier, and the names measurement units A-2 and B-1 are stored in the quality control object apparatus. Although not shown in the diagram, note that in the case of measurement unit A-2, measurement unit A-2 is stored in the apparatus identifier name, and measurement units A-1 and B-1 are stored in the quality control object apparatus. Similarly, in the case of measurement unit B-1, measurement unit B-1 is stored in the apparatus identifier name, and measurement units A-1 and A-2 are stored in the quality control object apparatus.

As shown in FIG. 9E the recording date item, control name item, lot number item, expiration date item, and target value item are included in the quality control sample information. The year/month/day the quality control sample was recorded is included in the record date item. Other items store information similar to the quality control sample information recorded in the RFID tag Q2. These items are recorded beforehand by the user using the quality control sample information stored in the RFID tag Q2 before quality control is performed. In FIG. 9E, for example, information of three quality control samples is stored in measurement unit A-1.

As shown in FIG. 9F, the analysis results include the measurement date/time item, apparatus identifier name item, control name item, and each measurement item (WBC, RBC). The control name used in quality control is stored in the control name item, and information similar to the analysis results of FIG. 9 (c) is stored in the other items. In FIG. 9F, for example, analysis results of a plurality of analyses are stored for the three measurement units A-1, A-2, B-1. Note that although the analysis results of measurement units A-2 and B-1 on 11/17 are written to the RFID tag Q2 of the quality control sample, the results of measurement unit A-1 are written to the hard disk 404 in FIG. 9F. Under the condition of the analysis results of measurement units A-2 and B-1 written to the RFID tag Q2 as shown in FIG. 9C, when the quality control measurement is performed by the measurement unit A-1, the analysis results of the measurement units A-2 and B-1 on 11/17 are written to the hard disk 404 of the measurement unit A-1.

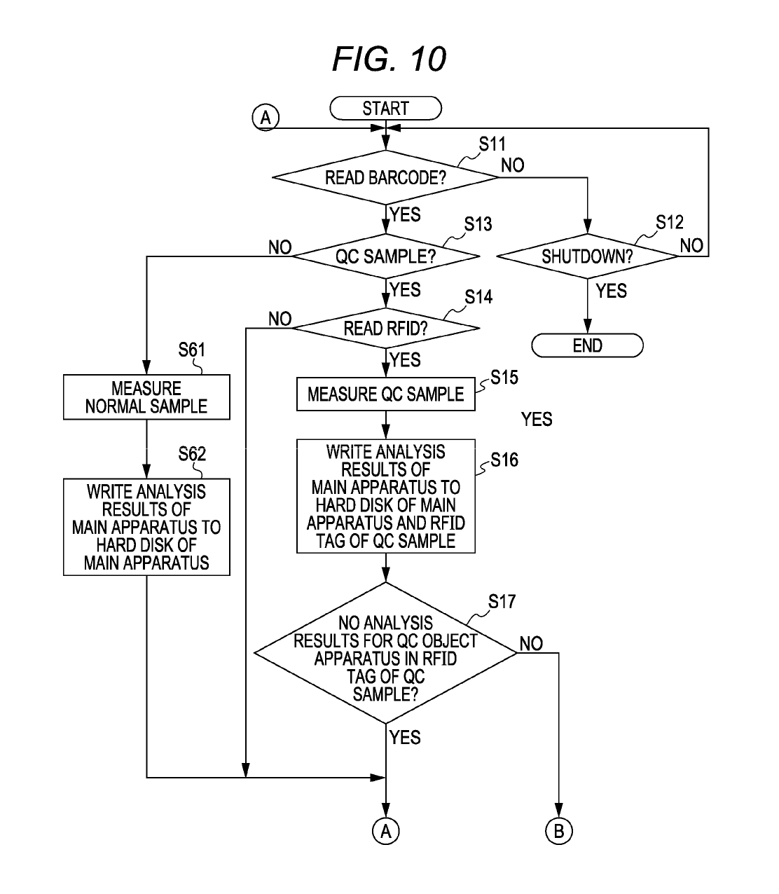

FIGS. 10 through 12 are flow charts showing the process of comparing the quality control measurements performed by the information processing unit 4 of the sample analyzer 1. Note that in the present embodiment the sample analyzer 1 that performed the quality control measurement is referred to as “main apparatus,” the sample analyzers 1 used for comparison of the quality control analysis results are referred to as “QC object apparatus.”

The CPU 401 of the information processing unit 4 waits to begin processing until the barcode readers B1 and B2 have read the barcode information (S11). That is, when the barcode information has not been read by the barcode readers B1 and B2(S11: NO), the CPU 401 returns the process to S11 insofar as the shutdown is not performed (S12: NO).

When the barcode information is read by the barcode readers B1 and B2 (S11: YES), the CPU 401 determines whether the container supplied to the measurement unit 3 contains a quality control sample based on the result of reading the barcode (S13). Note that although whether the sample is a quality control sample is determined based on the result of reading the barcode by the barcode readers B1 and B2 in the present embodiment, this determination may also be made according to user input or by the result of reading the RFID tag Q2.

When the container supplied to the measurement unit 3 does not contain a quality control sample (S13: NO), the normal sample measurement process is performed (S61), and the analysis result is written to the hard disk 404 of the main apparatus (S62). The CPU 401 thereafter returns the process to S11.

When the container supplied to the measurement unit 3 contains a quality control sample (S13: YES), the CPU 401 determines whether the RFID tag Q2 can be read by the RFID reader/writer RW (S14). When the RFID tag Q2 cannot be read by the RFID reader/writer RW (S14: NO), the CPU 401 returns the process to S11 without measuring the quality control sample. When the RFID tag Q2 can be read (S14: YES), the CPU 401 controls the measurement unit 3 to measure the quality control sample (S15). When the measurement of the quality control sample is completed, the analysis results are written to the hard disk 404 of the main apparatus and the RFID tag Q2 of the quality control sample container Q (S16). For example, when quality control is performed by the measurement unit A-1, the analysis results of the measurement unit A-1 which performed the quality control are stored both on the hard disk 404 and the RFID tag Q2 as shown in the fourth line of FIG. 9C and the sixth line of FIG. 9F. Note that when there is no empty space to which to write the analysis results at this time, the writing is performed sequentially from the oldest analysis results.

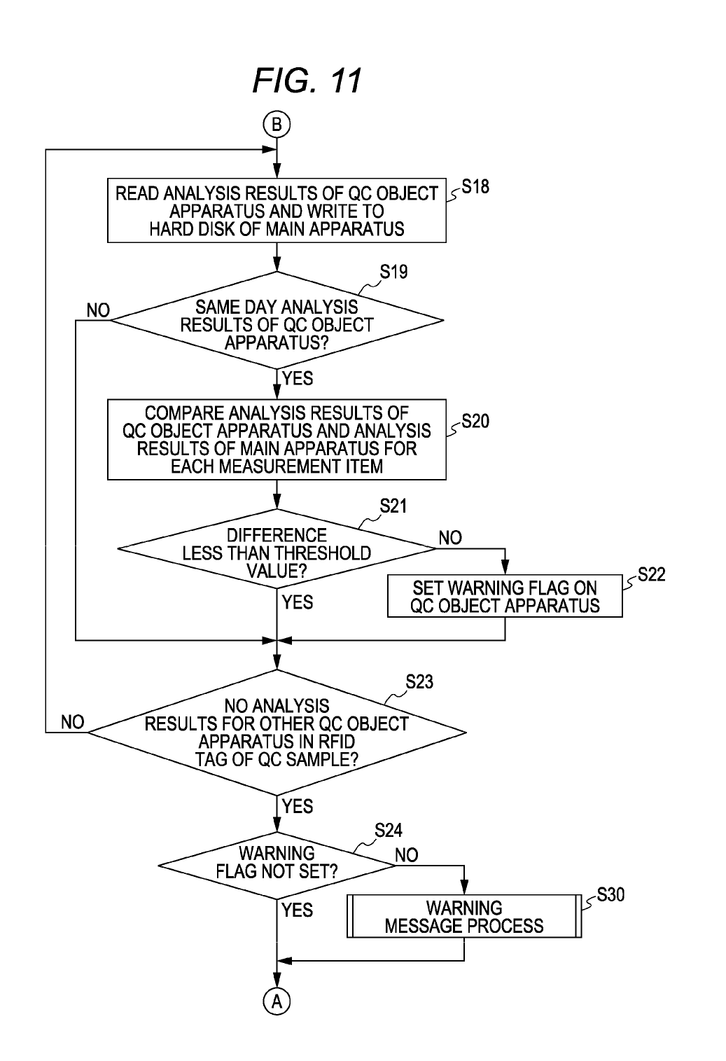

Returning to FIG. 10, the CPU 401 then determines whether the analysis results of the QC object apparatus set in the hard disk 404 has been stored in the RFID tag Q2 of the quality control sample (S17). When the analysis results of the QC object apparatus have not been stored in the RFID tag Q2 (S17: YES), the CPU 401 returns the process to S11 without comparing the analysis results of the quality control sample. When the analysis results of the QC object apparatus has been stored in the RFID tag Q2 (S17: NO), the process advances to S18 of FIG. 11.

Referring to FIG. 11, when the analysis results of the QC object apparatus are stored in the RFID tag Q2, the CPU 401 reads the analysis results of the QC object apparatus from the RFID tag Q2, and writes the read analysis results to the hard disk 404 of the main apparatus if analysis results identical to the read analysis results are not written to the hard disk 404 of the main apparatus (S18). Note that when analysis results identical to the read analysis results are already written to the hard disk 404 of the main apparatus, the CPU 401 advances the process to S20 without writing the read analysis results to the hard disk 404. For example, when the fourth line of analysis results in FIG. 9C are obtained in measurement unit A-1, the second and third lines of analysis results in FIG. 9C obtained in measurement units A-2 and B-1 are written to the hard disk 404 of the measurement unit A-1. Note that although the analysis results of the QC object apparatus are read from the RFID tag Q2 (S18) after the quality control sample has been measured by the main apparatus (S15, S16) in the above embodiment, the analysis results of the QC object apparatus also may be read from the RFID tag Q2 first, and thereafter the main apparatus may measure the quality control sample. In this way the step of performing measurement in the main apparatus and the step of reading the analysis results of the QC object apparatus can be suitable modified.

Returning to FIG. 11, the CPU 401 determines whether the analysis results have the same date as the date of the quality control measurement of the main apparatus in the read analysis results of the QC object apparatus (S19). When the analysis results do not have the same date (S 19: NO), the process advances to S23. When the analysis results have the same date (S19: YES), the CPU 401 compares the analysis results of the main apparatus and the newest analysis results among the analysis results of the QC object apparatus having the same date for each of the measurement items (WBC, RBC) (S20). The CPU 401 then calculates the difference of analysis results of the main apparatus and the newest analysis results for each measurement item, and determines whether the difference is below a predetermined threshold value (S21). Note that the threshold value is different for each measurement item. The process advances to S23 when the difference is below the threshold value for all measurement items (S21: YES), whereas a warning flag indicating a large difference between the measurement values of the main apparatus and the QC object apparatus is set for the QC object apparatus (S22) when a single difference exceeds the predetermined threshold value (S21: NO). For example, when the analysis results of the seventh line in FIG. 9C are obtained by the measurement unit B-1, the analysis results of the seventh line are compared to the analysis results of the sixth line of FIG. 9C which are the newest analysis results of the measurement unit A-2, that is the QC object apparatus. In this case the threshold value of the difference of the WBC measurement value is set at +/−400, and the difference of WBC measurement values of the measurement unit B-1 and the measurement unit A-2 is 600, which exceeds the threshold value and, hence, the warning flag indicating a large difference in measurement values is set in the comparison of with the measurement unit A-2.

Since the quality control sample degrades over time, there is concern that an accurate comparison may not be possible when the analysis result of the comparison object is old. Therefore, an accurate comparison can be made by using the newest analysis result, that is, the analysis result of the same day, as the comparison object from among the analysis results read from the RFID tag Q2.

Returning to FIG. 11, the CPU 401 then determines whether the analysis results of another QC object apparatus set in the hard disk 404 has been stored in the RFID tag Q2 of the quality control sample (S23). When analysis results of another QC object apparatus has been stored in the RFID tag Q2 (s23: NO), the process returns to S18. For example, when the analysis of the seventh line in FIG. 9C is obtained by the measurement unit B-1, the analysis results of the seventh line are compared to the analysis results of the measurement unit A-1 on the fourth line, and also compared to the analysis of the measurement unit A-2 on the sixth line.

When the analysis results of the other QC object apparatus are not stored in the RFID tag Q2 (S23: YES), the CPU 401 determines whether a warning flag is set for the QC object apparatus (S24). When a warning flag is set for any QC object apparatus (S24: NO), the CPU 401 performs a process to show on the display part 42 a warning message indicating that there is a wide difference in the analysis results between the main apparatus and a QC object apparatus (S30). However, when a warning flag is not set for any QC object apparatus (S24: YES), the CPU 401 ends the measurement and comparison processes of the quality control sample in the main apparatus without displaying a warning message, and the process returns to S11.

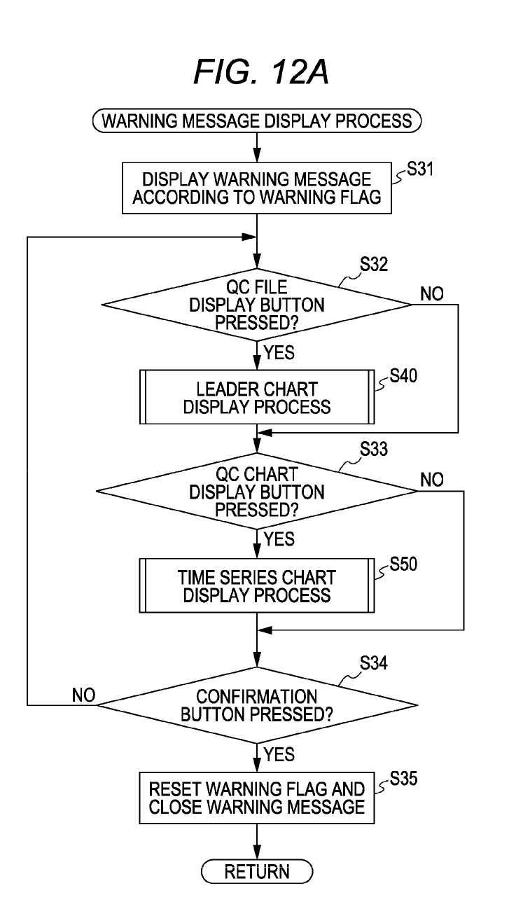

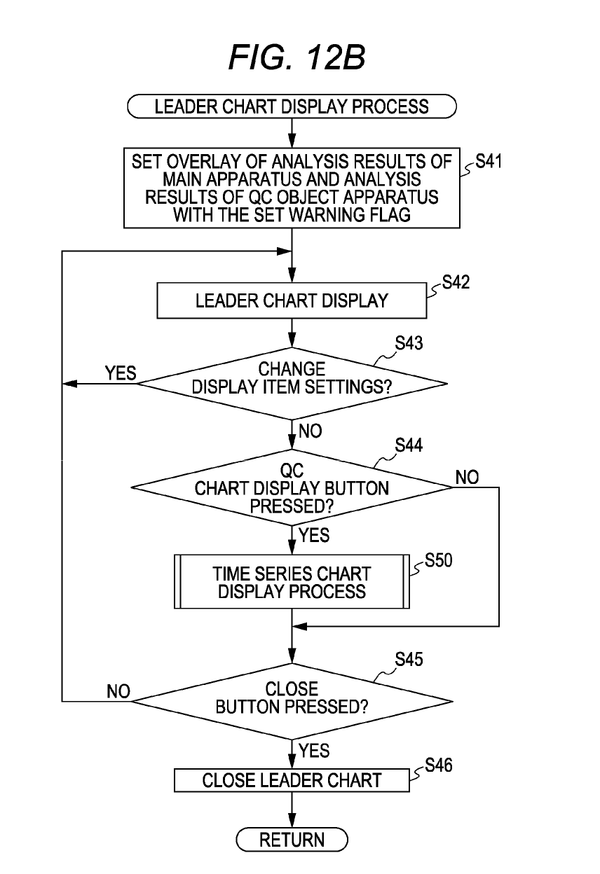

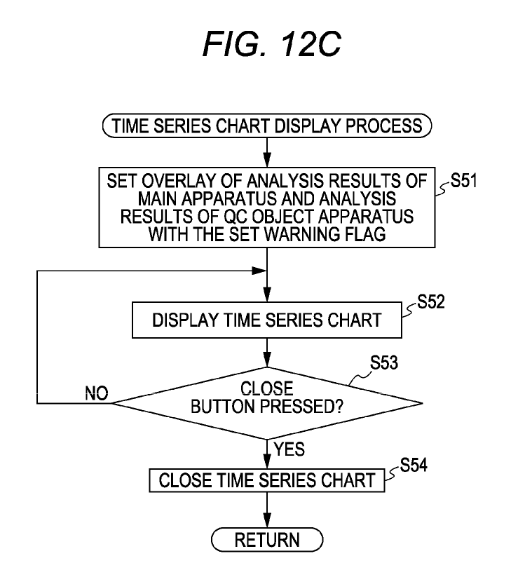

FIG. 12A is a flow chart of the warning message display process performed by the CPU 401, FIG. 12B is a flow chart of the leader chart display process performed by the CPU 401, and FIG. 12C is a flow chart of the time series chart display process performed by the CPU 401.

The warning message display process is described below referring to FIG. 12A. The CPU 401 displays the warning message on the display part 42 according to the presence/absence of a warning flag of the QC object apparatus set in S22 (S31).

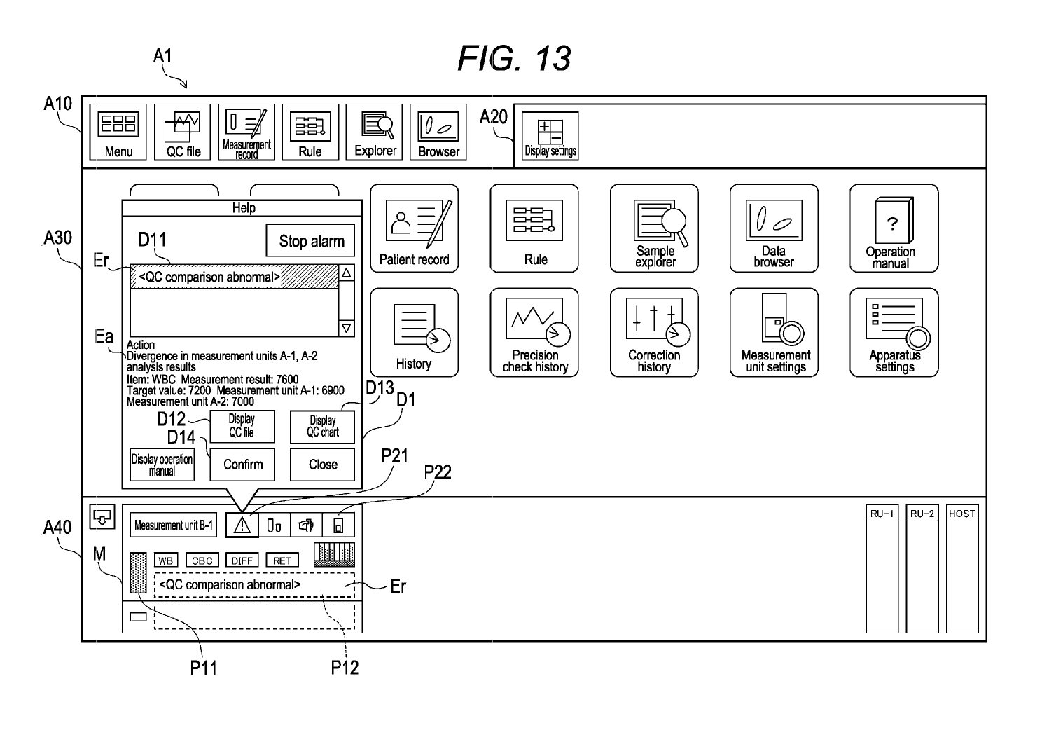

FIG. 13 shows an example of a warning message shown on the display part 42. Note that the warning message display example represents the situation when the difference between the quality control analysis results of the measurement unit B-1 and the quality control analysis results of the QC object apparatuses measurement units A-1 and A-2 exceeds the predetermined threshold (+/−400) as shown in the seventh line of FIG. 9C in the measurement unit B-1.

Referring to FIG. 13, the warning message Er is shown on the menu screen Al being displayed on the display part 42. The menu screen Al includes toolbars A10, A20, main region A30, and measurement operation region A40. Toolbars A10, A30, and main region A30 include a plurality of buttons. The user can issue various instructions to the information processing unit 4 by touching these buttons.

The measurement operation region A40 has an operation section M. The operation section M includes a status alert area P11, error/warning message display region P12, error/warning button P21 that has an error/warning icon, and an operation menu button P22.

The status alert area P11 displays green when the measurement unit 3 is operating normally, and displays red when the measurement unit 3 generates an error/warning. The error/warning message display region P12 shows an error/warning message when the an error/warning is generated by the measurement unit 3.

The error/warning button P21 is displayed when an error/warning is generated in the measurement unit 3. When an error/warning is generated, the error/warning button P21 is displayed together with a help dialog D1. The operation menu button P22 is used to open an operation menu screen (not shown in the drawing) capable of issuing instructions for various processes.

In FIG. 13, an abnormality is generated in measurement unit B-1 in comparison with the analysis results of the QC object apparatuses, measurement units A-1 and A-2. A warning message Er indicating an abnormality in the comparison of the quality control analysis results is shown in the error/warning message display region P12. The error/warning button P21 is also shown in the operation area M, and the help dialog D1 is shown at the top of the operation area M.

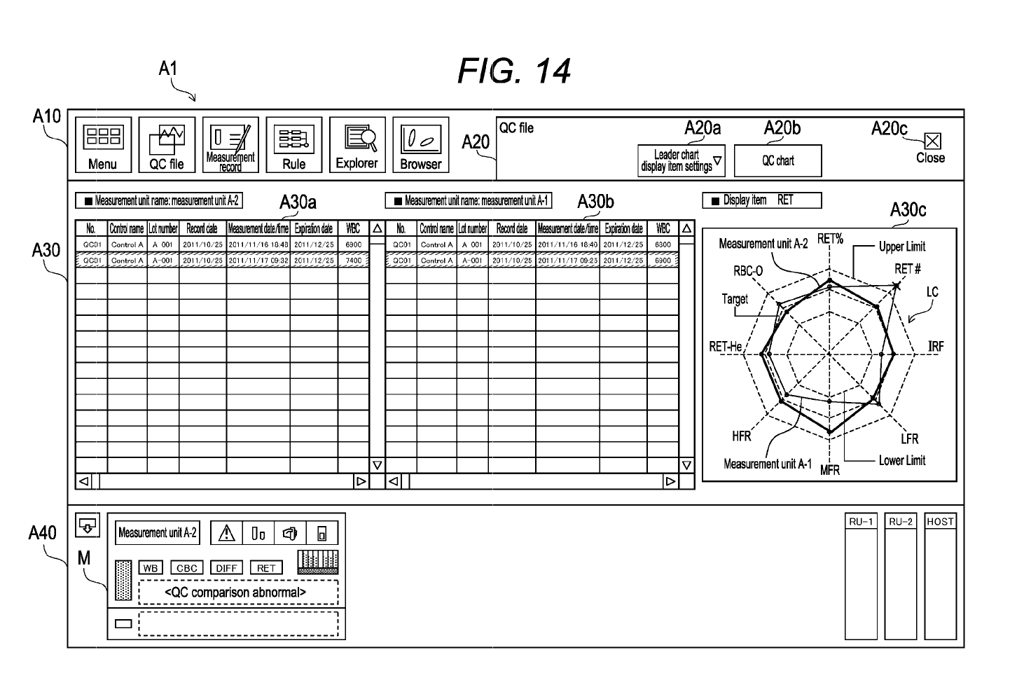

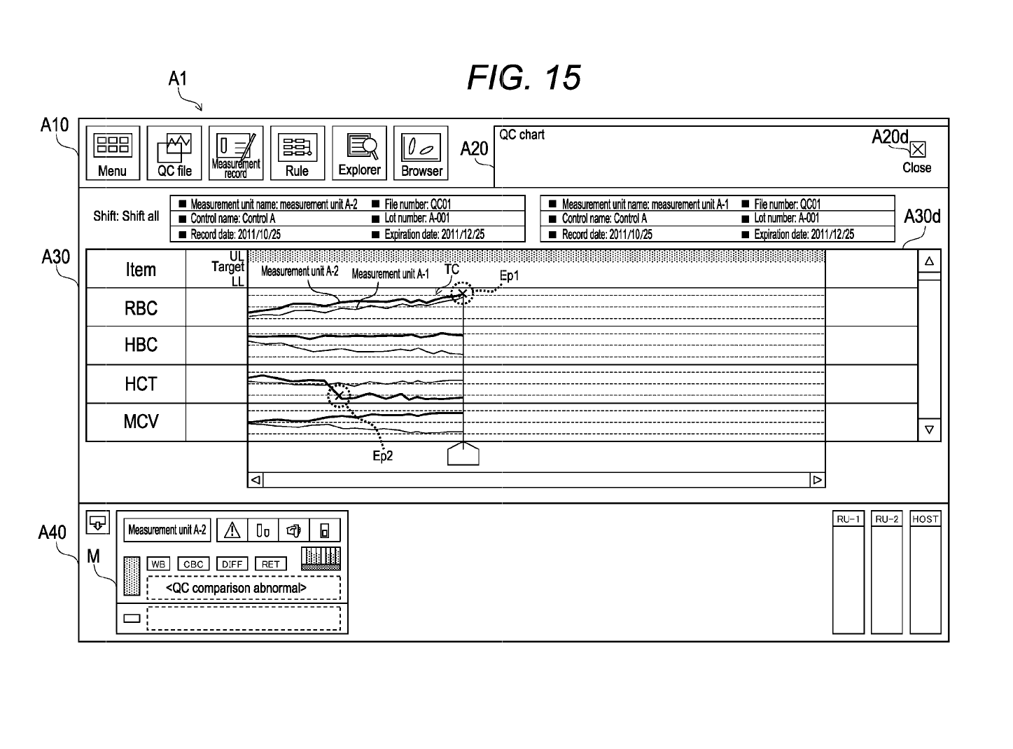

The help dialog D1 includes an error/warning message list D11, QC file display button D12, QC chart display button D13, and confirmation button D14. Note that hereinafter the screen shown in FIG. 14 displaying the leader chart and quality control analysis results, and the screen shown in FIG. 15 displaying the QC (quality control) file and time series chart TC, are referred to as the QC chart.

Returning to FIG. 13, the error/warning content is shown in the error/warning message list D11, and a plurality of error/warning items are shown when numerous error/warnings are generated simultaneously. In FIG. 13, a warning message Er indicating an abnormality in the comparison of the quality control analysis results is shown in the error/warning message list D11. Hence, the user can readily comprehend that a large discrepancy has occurred in the quality control analysis results between the main apparatus and the QC object apparatus. Since the warning message Er is automatically displayed when the difference between the quality control analysis results of the main apparatus and the analysis results of the QC object apparatus exceeds a predetermined threshold, the user is alerted that there is an abnormal comparison in the quality control analysis results soon after the quality control measurement.

An action message Ea indicating the content of the comparison result is displayed at the bottom of the error/warning message list D11. The action message Ea includes the name of the QC object apparatus that produced the divergent difference in analysis results that exceeded the threshold value, measurement item, measurement value of each apparatus, and the target value of the quality control sample. The user can readily comprehend the degree of divergence in the analysis results of the QC object apparatus by confirming the action message Ea. Since the target value is included in the display, the user can readily comprehend the amount of divergence in the relative values of the analysis results. Note that the display content of the action message Ea may omit part of the display items, or emphasize the most divergent analysis results in the display insofar as the display content shows the result of comparing the analysis results between the main apparatus and the QC object apparatus. The difference between each analysis result and the target value also may be shown.

Returning to FIG. 12, when the warning message is displayed, the CPU 401 determines the status (that is, whether pressed or unpressed) of the QC file display button D12 (S32), status of the QC chart display button D13 (S33), and status of the confirmation button D14 (S34) arranged above the help dialog D1.

When the QC file display button D12 is pressed (S32: YES), the CPU 401 performs a process to show thew leader chart of the quality control analysis results on the display part 42 (S40). When the QC chart display button D13 is pressed (S33: YES), the CPU 401 performs a process to show the time series chart of the time series quality control analysis results on the display part 42. When the confirmation button D14 is pressed (S34: YES), the CPU 401 resets the warning flag, and closes the warning message Er and the help dialog D1 shown on the display part 42 (S35). Hence, the warning message display process ends.

The leader chart display process is described below referring to FIG. 12B. The CPU 401 first sets the superposed targets of the analysis results of the main apparatus and analysis results of the QC object apparatus for which the warning flag is set (S41). The CPU 401 then generates a leader chart LC based on the analysis results of the superposed targets, and shows the generated leader chart LC on the display part 42.

FIG. 14 shows an example of a leader chart LC shown on the display part 42. Note that, unlike FIG. 13, the display example in FIG. 14 shows a difference in quality control analysis results that exceeds the threshold value between the QC object apparatus, measurement unit A-1, in the quality control analysis of the measurement unit A-2. That is, the screen shown in FIG. 14 is displayed when the QC chart display button D 13 is pressed while the same screen as FIG. 13 is shown on the display part 42 of the measurement unit A-2.

Referring to FIG. 14, the leader chart LC is shown in the leader chart display region A30c. The toolbar region A20 includes a display item setting button A20a, QC chart button A20b, and close button A20c.

The main region A30 is allocated an analysis result display region A30a, and the measurement results of the main apparatus (measurement unit A-2 in this case) are shown in this region. The main region A30 is also allocated an analysis result display region A30b, and this region shows the analysis results of the QC object apparatus (measurement unit A-1 in this case) which generated a difference that exceeded the threshold value relative to the analysis results of the main apparatus. When there are a plurality of QC object apparatuses which generate a difference that exceeds the threshold value in the analysis results relative to the main apparatus, the same number of analysis results display regions A30b are allocated to the main region A30. The analysis results display regions A30a and A30b are sized according to the number of allocated analysis results display region A30b.

The analysis results display region A30a also shows the past analysis results stored on the hard disk 404 in addition to the current quality control analysis results of the main apparatus. The analysis results display region A30b shows the past quality control analysis results of the QC object apparatus stored on the hard disk 404 of the main apparatus in addition to the analysis results of the QC object apparatus compared with the current analysis results of the main apparatus.

The leader chart object analysis results, that is, the analysis results of the main apparatus and the analysis results of the QC object apparatus which generated the warning flag, are highlighted among the analysis results shown in the analysis results display regions A30a and A30b.

The leader chart display region A30c shows predetermined display items of the relative relationships in the leader chart LC among the analysis results highlighted in the analysis results display regions A30a and A30b. In FIG. 14, the an example of the leader chart LC is shown with display items related to RET (reticulocytes). Thus, the leader chart based on the analysis results of the main apparatus and the leader chart LC of the analysis results of the QC object apparatus having the set warning flag are mutually superimposed and displayed. The user can thus promptly confirm thew measurement item/s causing the error/warning.

Note that the display items of the leader chart LC can be modified by the user operating the display items setting button A20a. When the display item setting button A20a is pressed, a pulldown menu is shown with the selectable display items as selection candidates. When the user selects a desired display item, the leader chart LC corresponding to the selected display item is shown in the leader chart display region A30c. For example, when the display item setting button A20a is pressed and the WBC display item is selected in the display condition of FIG. 14, the display of the leader chart display region A30c is switched from the leader chart with RET display items to the leader chart with WBC display items. The user can therefore confirm via these other display items the relative relationship of the analysis results of the main apparatus and the analysis results of the QC object apparatus which has the set warning flag.

As shown in FIG. 14, the upper limit and lower limit of the quality control sample target values are indicated by the dashed lines in the leader chart LC. The analysis results of the main apparatus and the analysis results of the QC object apparatus are displayed above the dashed line. In the display example of FIG. 14, the analysis results of the QC object apparatus (measurement unit A-1 in this case) are represented by the narrow line, and the analysis results of the main apparatus (measurement unit A-2 in this case) are represented by the thick line. An X mark icon is shown on the measurement item for which the quality control analysis results are outside the normal range (above the upper limit value or below the lower limit value).

The user therefore can readily see which apparatus and which measurement item diverges from the target value. In FIG. 14, for example, the user can confirm that the RET# (reticulocytes) measurement item of the measurement unit A-1, that is, the QC object apparatus, is far above the allowable upper limit value.

The degree of divergence of whichever analysis results can readily be confirmed even when the quality control analysis results are collected within the normal range (below the upper limit value and above the lower limit value) because the analysis results of the main apparatus and the QC object apparatus are displayed superimposed one over the other. In FIG. 14, for example, the values of the MFR (median reticulocyte fluorescence ratio) item of the measurement units A01 and A-2 are within the normal range, but a divergence can be confirmed. The user therefore can make an appropriate determination about a quality control re-measurement or replacement of the quality control sample. In the present embodiment, the user can closely investigate the analysis results and appropriately evaluate the analysis results by comprehending the occurrence of a discrepancy between the analysis results of the main apparatus and the QC object apparatus because the leader chart LC is displayed together with the screen showing the quality control sample analysis results.

Returning to FIG. 12, when the leader chart LC is displayed, the CPU 401 determines the status of the display item setting button A20a (S43), status of the QC chart button A20b (S44), and status of the close button A20c (S45) arranged above the toolbar region A20.

When the display item setting button A20a is pressed and the leader chart LC display item is changed (S43: YES), the CPU 401 shows the leader chart LC corresponding to the set display item in the leader chart display region A30c. The user thus suitably changes the display item to compare the quality control analysis results. Note that although only a single display item is shown in a single leader chart LC in the present embodiment, a plurality of display items may be shown in a plurality of aligned reader charts.

When the QC chart display button A20b is pressed (S44: YES), the CPU 401 performs a process to show the time series chart of the time series quality control analysis results on the display part 42 (S50). When the close button A20c is pressed (S45: YES), the CPU 401 closes the display of the leader chart LC and the quality control analysis results (S46). The leader chart display process therefore ends.

Note that in the screen of FIG. 14 the leader chart display object can be changed by the user selecting the analysis results that are not highlighted among the analysis results shown in the analysis results display regions A30a and A30b. For example, when the past analysis results are selected fro the QC object apparatus shown in the analysis results display region A30b, the leader chart of the selected past analysis results is shown in the analysis results display region A30b rather than the newest analysis results of the QC object apparatus. Hence, the user can compare and contrast the quality control analysis results of the main apparatus between the older analysis results without looking at the newest analysis results of the QC object apparatus, so as to more flexibly evaluate the quality control analysis results of the main apparatus.

The time series chart display process is described below referring to FIG. 12C. The CPU 401 first sets the superposed targets of the analysis results of the main apparatus and analysis results of the QC object apparatus for which the warning flag is set (S51). The CPU 401 then generates a time series chart TC based on the analysis results of superimposed targets, and shows the generated time series chart TC on the display part 42.

FIG. 15 shows an example of a time series chart TC shown on the display part 42. Note that, similar to the case shown in FIG. 14, the display example in FIG. 15 shows a difference in quality control analysis results that exceeds the threshold value between the QC object apparatus, measurement unit A-1, in the quality control analysis of the measurement unit A-2.

Referring to FIG. 15, the time series chart TC is shown in the time series display area A30d within the main region A30 of the main screen Al displayed on the display part 42. The toolbar area A20 includes the close button A20d.

The time series chart TC shows the set measurement items (RBC, HGB, HCT, MCV) in the time series chart display area A30d. The time series chart TC is a line graph in which the analysis results of the predetermined measurement items are arrayed in time series connected by a line, wherein the left end represents the oldest analysis results and the right end represents the newest analysis results. The time series chart TC is generated using the analysis results of the main apparatus (measurement unit A-2 in this case) recorded on the hard disk 404 and the analysis results of the QC object apparatus (measurement unit A-1 in this case) which has the warning flag set.

The time series chart TC shows the target value of the quality control sample, as well as the allowable upper limit and lower limit indicated by the dashed lines. The analysis results of the main apparatus and the analysis results of the QC object apparatus having the set warning flag are overlaid on the dashed lines. Note that, in FIG. 15, the analysis results of the QC object apparatus (measurement unit A-1) are represented by the narrow line, and the analysis results of the main apparatus (measurement unit A-2) are represented by the thick line. An X mark icon is shown on the measurement result at the time at which the quality control analysis results initially exceed the normal range (above the upper limit value or below the lower limit value).

The user therefore can readily verify which apparatus and which measurement item diverges from the target value. In the case shown in FIG. 15, for example, the user can confirm that the RBC (red blood cell count) measured value of the QC object apparatus, that is, measurement unit A-1, exceeds the upper limit value at time Ep1, and the HCT (hematocrit) measured value exceeds the lower limit value at time Ep2.

Even when the quality control analysis results are collected within the normal range (below the upper limit value and above the lower limit value), the degree of divergence between the main apparatus and the QC object apparatus can be readily verified by referring to the time series chart TC. For example, although the value of the HGB (hemoglobin) item is within the normal range, a certain divergence can be verified. The user therefore can make a more appropriate determination about a quality control re-measurement or replacement of the quality control sample.

The trend of change in measurement values of the main apparatus and other QC object apparatuses can be verified since the analysis results are shown in time series. The user therefore can make the evaluation below.

When comparing the RBC measurement item, the measured values of both measurement units A-1 and A-2 change so as to gradually rise. In this case the user can evaluate the possibility that the quality control sample has degraded from the obtained analysis results which show the same trend in both apparatuses. In the case of the HCT measurement item in FIG. 15, only the measured value of the measurement unit A-1 decreases markedly at time Ep2, whereas the measured value of the measurement unit A-2 maintains an unchanging trend from before time Ep2. In this case, the user evaluates the possibility of a detection system defect or malfunction in the measurement unit A-1 since the measured value shows a decreasing trend only in the measurement unit A-1.

Returning to FIG. 12C, when the time series chart is displayed, the CPU 401 determines the status of the close button A20d on the toolbar are A20 (S53). When the close button A20d is pressed (S53: YES), the CPU 401 closes the display of the time series chart (S54). The time series chart display process thus ends.

According to the present embodiment described above, the warning message Er, action message Ea, leader chart LC and time series chart TC are shown on the display part 42 based on the quality control sample analysis results of the main apparatus and the quality control sample analysis results obtained from the RFID tag Q2 by another sample analyzer. Therefore, printing and comparing the quality control sample analysis results for each analyzer is unnecessary, the work of the user is reduced, and quality control sample analysis results can be compared for each analyzer. Note that both analysis results can be compared using a simple structure since there is no need to install a system without an analyzer to obtain and process both analysis results separately.

According to the present embodiment, the user can easily become aware that the quality control sample analysis results of the main apparatus diverge from the allowable range from the quality control sample analysis results of the QC object apparatus since the warning message Er and the action message Ea are displayed.

According to the present embodiment, the user easily and smoothly becomes aware that the analysis results diverge from allowable values since the warning message Er and the action message Ea are displayed on the display part 42 which is monitored by the user to comprehend the analysis results.

According to the present embodiment, the results of the comparison with any QC object apparatus and whether a warning message Er is displayed can be known by confirming the action message Ea. Hence, the user knows the extent of the divergence of analysis results between the main apparatus and any QC object apparatus.

According to the present embodiment, suitable comparison is accomplished because the newest analysis results of the same day are compared from among the analysis results read from the RFID tag Q2.

According to the present embodiment, comparison of analysis results is easy since the analysis results of the measurement unit 3 and the analysis results of another QC object apparatus are displayed on the same screen as shown in FIGS. 14 and 15.

According to the present embodiment, the user is aware of the trend of the change between the analysis results of the main apparatus and the analysis results of another QC object apparatus since the quality control analysis results are shown in time series as indicated in FIG. 15. For example, if the trends of the change in both analysis results are the same, suitable evaluation can be made of the possibility of change (degradation) in the quality control sample.

According to the present embodiment, the user can compare the analysis results of the main apparatus and the analysis results of another QC object apparatus for each measurement item since the quality control analysis results are shown in leader chart as indicated in FIG. 14.

According to the present embodiment, the amount of divergence of the analysis results from an ideal value can be readily comprehended since the analysis results and the target values are shown together as indicated in FIGS. 14 and 15.

According to the present embodiment, the object of evaluation in another sample analyzer 1 serves since the analysis results of the quality control sample are stored in the RFID tag Q2.

According to the present embodiment, evaluation object sample analyzer 1 can be comprehended in another sample analyzer 1 since the analysis results of the quality control sample are written in the RFID tag Q2 together with the identification information that identifies the sample analyzer 1 which measured the quality control sample.

Although the present invention has been described above by way of an embodiment, the present invention is not limited to this embodiment.

For example, although the above embodiment is described by way of example of blood as an object to be measured, urine also be an object to be measured. That is, the present invention may be applied to an analyzer for analyzing urine, and the invention also may be applied to a clinical analyzer for examining other clinical specimens.

Although the difference between two analysis results in excess of a threshold value is set as a warning condition in the comparison of quality control analysis results of a main apparatus and a QC object apparatus in the above embodiment, various warning conditions may be set as the warning condition, that is, when the difference between the analysis results of the main apparatus and the mean value of analysis results of a plurality of QC object apparatuses is in excess of a threshold value, when the difference between the analysis results of the main apparatus (measurement unit A-2) from past history and the analysis results of a QC object apparatus (measurement unit A-1) gradually broadens as in the MCV measurement item (mean corpuscular volume) of FIG. 15 and the like. The threshold value need not be a fixed value and may be, for example, obtained by multiplying a target value by a predetermined ratio.

Although the analysis results of the main apparatus and the QC object apparatus are stored on both the hard disk 404 of the sample analyzer 1 and the RFID tag Q2 in the above embodiment, the analysis results also may be stored only on the hard disk 404 of the sample analyzer 1, or stored only in the RFID tag Q2. Although the CPU 401 reads the content of the hard disk 404 and displays the analysis results of FIGS. 14 and 15 in the above embodiment, when the analysis results are only stored on the RFID tag Q2, the CPU 401 may reads the content of the RFID tag Q2 and display the analysis results of FIGS. 14 and 15. Note that storing the quality control analysis results on the hard disk 404 of the sample analyzer 1 is ideal for confirming the quality control history even without reading the RFID tag Q2. For example, when the history of the QC object apparatus is pre-stored on the hard disk 404 of the sample analyzer 1, it is possible to compare quality control analysis results with past analysis results of the QC object apparatus even when the RFID tag Q2 cannot be read in flow chart of FIG. 10 (S14: NO).

Although quality control sample information is stored on the hard disk 404 of the sample analyzer 1 as shown in FIG. 9E of the above embodiment, the RFID tag Q2 has a storage capacity to be capable of a certain amount of data. In this case, it is suitable to use the quality control sample information read from the RFID tag Q2 in the comparison and display of the QC object apparatus. Thus, the capacity of the hard disk 404 may be reduced, and the work of the user in recording the quality control sample information can be reduced.

Although a sample analyzer 1 having a single measurement unit 3 is used in the example of the above embodiment, the present invention also may be applied to sample analyzers having, for example, two or more measurement units. the present invention also may be applied to a sample analyzer that lacks a transporting unit 2, wherein the user manually supplies the sample container to the measurement unit. In this case, a handheld type RFID tag reader/writer reads and writes the quality control analysis results to/from the RFID tag Q2 with an optional timing of the user.

Although an abnormality in the comparison results of the quality control is indicated to the user by displaying a warning message Er on the display part 42 in the above embodiment, a speaker (audio warning) and light emitter (warning by light) also may be used in addition to displaying o the display part 42. The background color also may be changed from the normal color, or flash on the display part 42.

Although, in the above embodiment, the information indicating that the threshold value was exceeded by the difference between the analysis results of the QC object apparatus and the analysis results of the apparatus performing quality control is displayed in text via the action message Ea of FIG. 13, the information indicating this difference exceeds the threshold value also may be provided by displaying an X mark, symbol, graphic, flashing a predetermined image or the like on the item exceeding the threshold value in FIG. 15.

Although the analysis results of each apparatus are shown via numerical values, the leader chart LC, and the time series chart TC in the above embodiment, the analysis results also may be shown by graphics of lines and dots, or combining graphic and text, and symbols. As shown in FIG. 14, a plurality of analysis results may be overlaid on a common region, or displayed in separate regions. Analysis results are preferably displayed in overlay to allow comparison of the analysis results.

Although the target values are shown in numerical values in the action message Ea, and shown by dashed lines in the leader chart LC and the time series chart TC of the above embodiment, the target values also may be shown as graphics of dots and lines, or combinations of such graphics and text, and symbols.

Although a passive type RFID tag is used as the RFID tag Q2 in the above embodiment, an active type RFID tag which is self-powered by an internal power source and emits radio waves also may be used. In this case, when the quality control sample container Q is disposed at the RFID tag reading position P5, the RFID tag Q2 adhered to the quality control sample container Q emits radio waves and the information recorded in the RFID tag Q2 is read by the RFID reader/writer RW.

Although the quality control measurement and comparison are performed using a quality control sample prepared for use in quality control in the above embodiment, whole blood from a healthy human may also be used to perform quality control. In this case, the whole blood of a healthy human is collected in the quality control sample container Q. The RFID tag Q2 also may have an empty area to which the analysis results may be written without storing the quality control sample information.

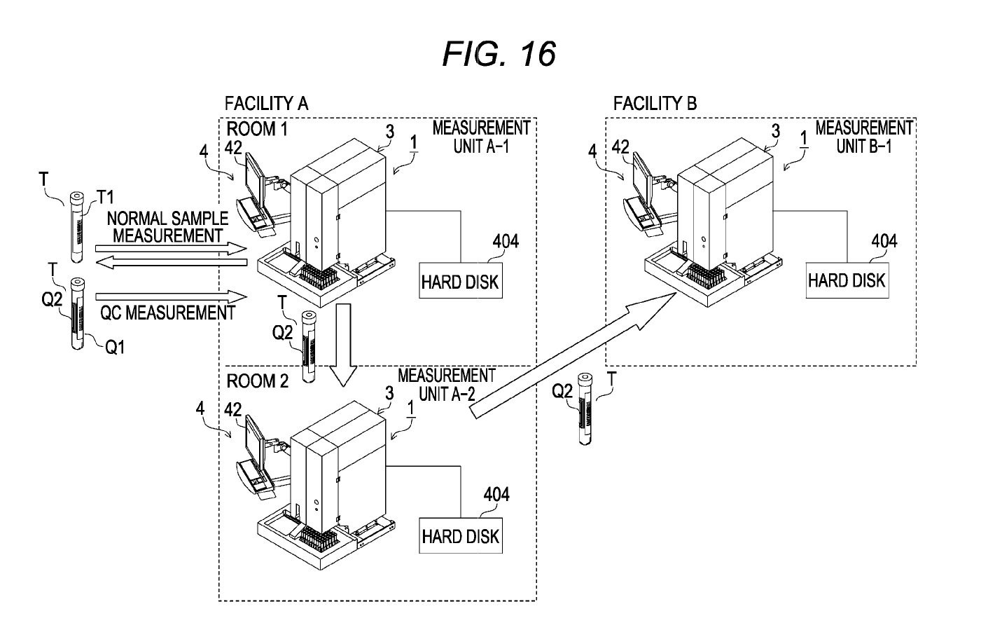

In this case, a normal sample measurement process is performed by a sample analyzer 1 provided with a measurement unit A-1 in a room 1 of facility A, and a determination is made as to whether the analysis results are in the normal range as shown in FIG. 16. If the analysis results are in the normal range, the barcode label T1 adhered to a sample container T is pasted on the quality control barcode label Q1, then the RFID tag Q2 is affixed to the sample container T. Thereafter, quality control is executed by the sample analyzer 1 provided with the measurement unit A-1 using the sample container T. Hence, the analysis results are stored in the RFID tag Q2. This sample also may be used as the quality control sample by the sample analyzer 1 in room 2 of facility A, the sample analyzer 1 of facility B, or a plurality of other sample analyzers 1. Quality control comparison and contrast therefore can be performed among a plurality of apparatuses via the same process flow as in the above embodiment. Quality control comparison and contrast also can be performed among a plurality of apparatuses without using expensive quality control samples.

Note that in this case the analysis results of the quality control performed using the measurement unit A-1 also may be used as the target value in the other sample analyzers 1. That is, the analysis results stored initially in the RFID tag Q2 may be used as the target values. In this case the initially stored analysis results are recorded on the hard disk 404 as the target value from among the analysis results read from the RFID tag Q2 by another sample analyzer 1, and this analysis result may be used as the analysis results of another sample analyzer being compared to the quality control analysis results performed by the sample analyzer 1 itself. When the initially stored analysis results are used as the target values, the sample apparatus 1 that performed the initial quality control preferably has high performance and reliability. A sample container barcode label T1 is adhered over the quality control barcode label Q1 since a normal sample is used for quality control in this case; however, quality control also may be executed in manual mode by another sample analyzer 1 without replacing the barcode label.

Note that the present invention is not limited to the above described embodiments and may be variously modified insofar as such modification are within the scope of the claims.

What is claimed is:

1. A sample analyzer comprising:
a sample analysis unit which analyzes a sample;
a reading unit which reads information from a storage medium attached to a container that contains a quality control sample, the storage medium being capable of storing an analysis result obtained by a sample analyzer;
a display; and
a controller which displays on the display an analysis result obtained by another sample analyzer and read from the storage medium and an analysis result obtained by the sample analysis unit, when the storage medium stores the analysis result obtained by the other sample analyzer.

2. The sample analyzer of claim 1, wherein
the controller displays on the display a screen which shows the analysis results obtained by the sample analyzer and the analysis results read from the storage medium.

3. The sample analyzer of claim 2, wherein
the controller displays on the display a screen which shows the time series of a plurality of analysis result at different analysis times obtained by the sample analyzer and a plurality of analysis results at different times obtained by another sample analyzer.

4. The sample analyzer of claim 3, wherein
the controller displays on the display a screen which shows a first line graph in which the plurality of analysis results obtained by the other sample analyzer are connected by a line in a time series, and a second line graph in which the plurality of analysis results obtained by the sample analyzer are connected by a line in a time series.

5. The sample analyzer of claim 2, wherein
the analysis results obtained by the sample analyzer and the analysis results obtained by the other sample analyzer respectively comprise analysis values for a plurality of items; and
the controller displays on the display a screen showing a leader chart indicating analysis values included in the analysis results obtained by the sample analyzer for a plurality of items, and analysis values included in the analysis results read from the storage medium.

6. The sample analyzer of claim 2, wherein
the controller displays on the display a screen which shows analysis results read from the storage medium, analysis results obtained by the sample analyzer, and target values of the analysis results of the quality control sample.

7. The sample analyzer of claim 1, wherein
the controller displays on the display a specific warning information when the relationship between the analysis results read from the storage medium and the analysis results obtained by the sample analyzer match a predetermined condition.

8. The sample analyzer of claim 7, wherein
the controller displays on the display warning information when the difference between the analysis results read from the storage medium and the analysis results obtained by the sample analyzer exceeds a predetermined threshold value.

9. The sample analyzer of claim 1, wherein
the controller gives a specific warning when the relationship between the analysis results read from the storage medium and the analysis results obtained by the sample analyzer match a predetermined condition.

10. The sample analyzer of claim 7, wherein
the controller does not display on the display warning information when the relationship between the analysis results read from the storage medium and the analysis results obtained by the sample analyzer do not match a predetermined condition.

11. The sample analyzer of claim 1, wherein
the storage medium stores analysis results obtained by another sample analyzer, and identification information which identifies the other sample analyzer that obtained the analysis results;
the controller displays on the display information identifying the other sample analyzer based on the identification information read from the storage medium.

12. The sample analyzer of claim 1, wherein
the storage medium stores the analysis results obtained by the other sample analyzer, and the date on which the analysis results were obtained;
the controller displays on the display the newest analysis results within a predetermined period among the analysis results read from the storage medium, and the analysis results obtained by the other sample analyzer.

13. The sample analyzer of claim 12, wherein
the controller displays on the display the analysis results obtained by the sample analyzer, and the analysis results obtained on the same day, that is the newest analysis results of the sample analyzer from among the analysis results read from the storage medium.

14. The sample analyzer of claim 1, wherein
the reading unit is configured to write information to the storage medium;
the controller writes the analysis results obtained by the sample analyzer to the storage medium through the reading unit.

15. The sample analyzer of claim 14, wherein
the controller writes the analysis results obtained by the sample analyzer, and the identification information which identifies the sample analyzer that obtained the analysis results to the storage medium through the reading unit.

16. The sample analyzer of claim 1, wherein
the storage medium is an electronic tag capable of non-contact communication with the reading unit.

17. The sample analyzer of claim 1, wherein
the sample analyzer is configured to analyze blood cells in blood held in a container;
the quality control sample is a control blood containing predetermined amounts of blood cell components obtained by preparing blood collected from a living body, and artificially prepared pseudo blood cell components.

18. A sample container for quality control, comprising:
a container for holding a quality control sample; and
a storage medium which is readable and writable for analysis information of the quality control sample held in the container.

19. The quality control sample container of claim 18, wherein
the storage medium is an electronic tag capable of non-contact communication with the reading unit.

20. The quality control sample container of claim 18, wherein
the container holds the quality control sample.

21. A quality control method for performing quality control of a sample analyzer, comprising:
reading from a storage medium a first analysis result obtained by a first sample analyzer analyzing a quality control sample held in a container;
obtaining a second analysis result from a second sample analyzer which analyzes the quality control sample held in the container and is different from the first sample analyzer; and
displaying the first analysis result and the second analysis result on a display of the second sample analyzer.
